(19) United States
(12) Patent Application Publication
(10) Pub. No.: US 2022/0133647 A1
(43) Pub. Date: May 5, 2022

(54) LIQUID GUAIFENESIN COMPOSITIONS WITH STABLE EXTENDED RELEASE PROFILES

(71) Applicant: Avadel Legacy Pharmaceuticals, LLC, Chesterfield, MO (US)

(72) Inventors: Pierre DANNER, Chesterfield, MO (US); Alexandre DROGOZ, Chesterfield, MO (US); Alain CONSTANCIS, Chesterfield, MO (US)

(21) Appl. No.: 17/434,996

(22) PCT Filed: Feb. 28, 2020
(86) PCT No.: PCT/US20/20410

§ 371 Date: Aug. 30, 2021

Related U.S. Application Data
(60) Provisional application No. 62/812,708, filed on Mar. 1, 2019.

(51) Int. Cl. A61K 31/09; A61K 9/50; A61K 47/32; A61K 47/38; A61K 47/02; A61K 47/12; A61K 47/26

(52) U.S. Cl. CPC A61K 31/09; A61K 9/5073; A61K 47/32; A61K 47/38; A61K 47/26; A61K 9/5047; A61K 47/02; A61K 47/12; A61K 9/5026

(57) ABSTRACT

The present disclosure provides drug-containing sustained-release particles that have minimal loss of the drug from the particle when the particle is formulated as a suspension in a liquid phase that is saturated by the drug.

### CROSS-REFERENCE TO RELATED APPLICATIONS

This application claims priority to U.S. Provisional Application No. 62/812,708, filed Mar. 1, 2019.

### FIELD

The present disclosure generally relates to liquid guaifenesin compositions with stable extended release profiles.

### BACKGROUND

Guaifenesin may diffuse out of a sustained-release (SR) particle when the particle is dispersed in a continuous phase and stored as a suspension for an extended period of time. The diffusion causes a change in the dissolution profile of the SR particle and, when formulated in a continuous phase with an immediate-release (IR) component, the diffusion also increases the IR fraction.

Therefore, there is a need for pharmaceutical dosage forms of guaifenesin SR particles dispersed in a continuous, liquid phase having improved stability over an extended period of time.

### SUMMARY

In an aspect, the present disclosure encompasses a sustained-release particle comprising a core, a first coating, and a second coating. The core may include guaifenesin. The first coating may include about 90 wt % or more of one or more hydrophilic polymers and cover the core to form an intermediate particle. The coating ratio of the first coating may be about 5 wt % to about 15 wt % of the intermediate particle. The second coating may include about 80 wt % or more of one or more hydrophobic, sustained-release polymers, less than about 15 wt % of a plasticizer, and the second coating covers the first coating. After storage of the particle as a suspension, for at least three months at about 30° C. and about 65% relative humidity, the particle may have a stable in vitro dissolution profile.

In another aspect, the present disclosure provides a pharmaceutical composition having a plurality of the sustained release particles dispersed in a liquid phase. The composition may include at least about 10% of the guaifenesin in the form of an immediate-release (IR) fraction. The amount of guaifenesin in 1 ml of the composition may be about 60 mg to about 240 mg. After storage of the composition for at least three months at about 30° C. and 65% relative humidity, the composition may have a stable in vitro dissolution profile.

In other aspects, the present disclosure provides a method of treating coughing, symptoms of coughing, nasal discharge, congestion or sneezing associated with a cold, flu or an allergy by administering to a human subject in need thereof a single dose of the pharmaceutical composition.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1A-D graphically depict the results of in vitro dissolution tests (USP II, 37° C., phosphate buffer, pH6.8). The percent of drug released into the dissolution medium is graphed on the y-axis versus time (hours, x-axis). The samples tested are suspensions of guaifenesin particles (20% IR/80% SR) stored at 25° C. with 60% relative humidity (FIGS. 1A and 1C) or 30° C. with 65% relative humidity (FIGS. 1B and 1D) for t=0 (solid green line), t=3 months (dotted blue line), t=4.5 months (dashed blue line), t=6 months (dashed red line), or t=12 months (solid blue line). A t=0 suspension is a suspension 5 days after manufacturing of the suspension. In FIGS. 1A and 1B, the particles have no binder in the core, a PVA coating (CR 10 wt %), and a sustained-release coating of EC100/polyvinylpyrrolidone/Kolliphor RH40/CO (89/3/4/4; CR 15 wt %). In FIGS. 1C and 1D, the particles have a sustained-release CR of 20 wt % but are as otherwise described in FIGS. 1A and 1B. In FIGS. 1A-D, the in vitro dissolution profile of the suspension after storage does not substantially differ from the in vitro dissolution profile of the initial solution.

FIG. 2A-C graphically depict the results of in vitro dissolution tests (USP II, 37° C., phosphate buffer, pH 6.8). The percent of drug released into the dissolution medium is graphed on the y-axis versus time (hours, x-axis). The samples tested are suspensions of guaifenesin particles (20% IR/80% SR) stored at 30° C. with 65% relative humidity. The liquid phase contains 1% PVP. A t=0 suspension is a suspension 5 days after manufacturing of the suspension. In FIG. 2A, the particles have no binder in the core, a PVA coating (CR 10 wt %), and a sustained-release coating of EC100/polyvinylpyrrolidone/Kolliphor RH40/CO (89/3/4/4; CR 15 wt %). In FIG. 2B, the particles have a sustained-release CR of 20 wt % but are as otherwise described in FIG. 2A. In FIG. 2C, the particles lack a PVA coating and have a sustained-release CR of 15 wt %, but are as otherwise described in FIG. 2A. After storage the in vitro dissolution profile of the suspensions with particles containing a PVA coating do not substantially differ from the in vitro dissolution profile of the initial suspension.

FIG. 3A-B graphically depict the results of in vitro dissolution tests (USP II, 37° C., phosphate buffer, pH 6.8). The percent of drug released into the dissolution medium is graphed on the y-axis versus time (hours, x-axis). The samples tested are dry particles stored at 40° C. with 75% relative humidity for t=0 (solid purple line), t=1 month (dotted red line), t=3 months (dashed red line), or t=6 months (dashed dark red line). A t=0 suspension is a suspension 5 days after manufacturing of the suspension. In FIG. 3A, the particles have no binder in the core, a PVA coating (CR 10 wt %), and a sustained-release coating of EC100/polyvinylpyrrolidone/Kolliphor RH40/CO (89/3/4/4; CR 20 wt %). In FIG. 3B, the particles lack a PVA coating but are as otherwise described in FIG. 3A.

FIG. 4 graphically depicts the results of in vitro dissolution tests (USP II, 37° C., phosphate buffer, pH 6.8). The samples tested are suspensions of guaifenesin particles (20% IR/80% SR) stored at 30° C. with 65% relative humidity for t=0 (solid green line), t=1 month (dotted orange line), t=2 months (dashed orange line), t=3 months (dashed orange line), or t=6 months (dashed red line). A t=0 suspension is a suspension 5 days after manufacturing of the suspension. The particles have a binder in the core, a PVA coating (CR 10%), and a sustained-release coating of EC100/polyvinylpyrrolidone/Kolliphor RH40/CO (89/3/4/4; CR 20 wt %).

FIG. 5 graphically depicts the results of in vitro dissolution tests (USP II, 37° C., phosphate buffer, pH 6.8). The samples tested are suspensions of guaifenesin particles (10% IR/90% SR) stored at 30° C. with 65% relative humidity for t=0 (solid green line), t=1 month (dotted orange line), t=2 months (dashed orange line), t=3 months (dashed orange line), t=4.5 months (dash-dot-dash orange line), or t=6 months (dashed red line). A t=0 suspension is a suspension 5 days after manufacturing of the suspension. The particles have a binder in the core, a PVA coating (CR 10%), and a sustained-release coating of EC100/polyvinylpyrrolidone/Kolliphor RH40/CO (89/3/4/4; CR 20 wt %).

FIG. 6A-B graphically depict the results of in vitro dissolution tests (USP II, 37° C., phosphate buffer, pH 6.8). The samples tested are suspensions of guaifenesin particles stored at 30° C. with 65% relative humidity for t=0 (solid green line), t=1 month (dotted orange line), t=2 months (dashed orange line), t=3 months (dashed orange line), or t=6 months (dashed red line). A t=0 suspension is a suspension 1 day after manufacturing of the suspension. The particles have a binder in the core, a PVA coating (CR 10%), and a sustained-release coating of EC45/polyvinylpyrrolidone/Kolliphor RH40/CO (80/2/2/16; CR 15 wt %). In FIG. 6A the suspension is 5% IR and 95% SR particles. In FIG. 6B, the suspension is 20% IR and 80% SR particles.

FIG. 7A-B graphically depict the results of in vitro dissolution tests (USP II, 37° C., phosphate buffer, pH 6.8). The samples tested are suspensions of guaifenesin particles (20% IR/80% SR) stored at 30° C. with 65% relative humidity for t=0 (solid green line), t=1 month (dotted orange line), t=2 months (dashed orange line), t=3 months (dashed orange line), or t=6 months (dashed red line). A t=0 suspension is a suspension 5 days (FIG. 7A)/3 days (FIG. 7B) after manufacturing of the suspension. The particles have a binder in the core, a PVA coating (CR 10%), and a sustained-release coating of EC45/Cellulose acetate butyrate/polyvinylpyrrolidone/Kolliphor RH40/CO (66/19/3/4/8; CR 15 wt %). In FIG. 7A, the suspension includes 1% PVP and the suspension in FIG. 7B does not include PVP.

### DETAILED DESCRIPTION

The present disclosure provides drug-containing sustained-release particles that have minimal loss of the drug from the particle when the particle is formulated as a suspension in a liquid phase that is saturated by the drug. The present disclosure also provides compositions that are suspensions comprising a plurality of drug-containing sustained-release particles dispersed in a liquid phase that is saturated by the drug. Several technical challenges may be faced when formulating a composition comprising sustained-release (SR) guaifenesin particles suspended in a liquid phase that also contains solubilized guaifenesin and crystals of guaifenesin. For example, crystals may grow by adsorption of solubilized guaifenesin while causing diffusion of guaifenesin from SR particles to re-saturate the continuous phase. Therefore, these compositions have a short shelf life due to loss of guaifenesin in the particles. An advantage of the compositions disclosed herein is that they are stable when stored for weeks or months, even at temperatures above 20-25° C. (i.e., room temperature). Other aspects of the particles and compositions of the present disclosure are described more thoroughly below.

Several definitions that apply throughout this disclosure will now be presented. As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention. For example, a particle consisting essentially of a drug-containing core and a sustained-release coating could not include a second, unrecited coating unless that coating did not materially affect the drug-release profile imparted to the particle by the sustained-release coating.

A “coating” is a composition that is layered over something, for example, a drug-containing core. As used herein, the terms “coating” and “layer” may be used interchangeably. The amount of each component in a coating is expressed as a percent of the total weight of the coating. For example, an ethylcellulose/polyvinylpyrrolidone/castor oil (80 wt %/10 wt %/10 wt %) coating is a coating consisting of 80 weight percent (wt %) ethylcellulose, 10 wt % polyvinylpyrrolidone, and 10 wt % castor oil. If units are omitted (e.g., 80/10/10), it is understood that the amounts indicated are weight percent. Particles of the present disclosure may have one or more coatings. As such, a coating may or may not be adjacent to the particle's core and may or may not be on the outside of the particle (i.e., an outer coating).

The term “coating ratio” refers to the weight of a coating applied to a particle. Coating ratio is abbreviated as “CR.” The coating's weight is expressed as a percent of the total weight of the particle after the coating has been applied. For example, a coating ratio of 25 wt % indicates that a coating was applied to a particle (whether the particle was initially uncoated or previously coated) that accounts for 25% of the coated particle's weight.

Particles of the present disclosure have a drug-containing core. The term “drug-containing core” refers to the innermost portion of the particle that contains a drug. In other embodiments, the drug-containing core may be a drug-coated pellet or bead.

The term “immediate release,” as used herein, describes the rate of drug release from a composition. An immediate-release composition, or a composition with an immediate-release fraction, allows for drug to be released immediately upon administration. An “immediate-release fraction” refers to the amount of the non-modified drug that is initially dissolved/dispersed in the suspension. A “rapid-release fraction” refers to the total amount of the drug that is released within the first 30 minutes of an in vitro dissolution test, and is typically expressed as a percent of the total drug content (w/w). For example, the rapid-release fraction may include the fraction of the drug initially in the immediate-release fraction (that is the non-modified drug initially added in the composition) and the fraction of the drug initially in the sustained-release particles that is released in the first 30 minutes. For compositions that are unstable in suspension, the rapid-release fraction can increase dramatically over time.

The term “sustained release,” as used herein, describes the rate of drug release from a composition. A sustained-release composition, a composition with a sustained-release fraction, a sustained-release particles, etc., allow for drug to be released for an extended period upon administration. A “sustained-release fraction” refers to the amount of the drug coated with one or more coating layers that is initially dispersed in the suspension. This fraction is released over an extended period of time (e.g. several hours in an in vitro dissolution test), and is typically expressed as a percent of the total drug content (w/w).

The term “similarity factor (f₂)” refers to a logarithmic reciprocal square root transformation of the sum of squared error, where

f₂=50·log{[1+(1/n)Σₜ₌₁ⁿ(Rₜ−Tₜ)²]^{-0.5}·100}.

An f₂ value provides a measurement of the similarity between two curves (e.g., two in vitro dissolution curves). Generally, f₂ values of 50 or greater (i.e., 50-100) ensure sameness or equivalence of two curves. See, for example, www.fda.gov/downloads/drugs/guidancecomplianceregulatoryinformation/guidances/ucm070237.pdf, which is hereby incorporated by reference.

A “suspension of particles,” as used herein, refers to a plurality of drug-containing particles dispersed in a liquid phase. The particles may or may not be homogenously dispersed in the liquid phase.

A “continuous phase” or “liquid phase” as used herein, refers to the fluid or liquid phase of the suspension in which the particles are dispersed. The continuous phase may be saturated by the drug. “Saturated by the drug” as used herein refers to the amount of drug in the continuous phase being at least at the saturation limit of the drug in the continuous phase at room temperature.

A “stable particle,” as used herein, refers to a particle that has a stable in vitro dissolution profile after storage. Preferably, a stable particle may also be stored as a suspension without a significant amount of the drug from the particle appearing in the liquid phase of the suspension, for example as evidenced by the absence of a significant change in the suspension's immediate-release fraction.

A “stable suspension,” as used herein, refers to a suspension of drug-containing particles that may be stored for a period of time without a significant change to the suspension's in vitro dissolution profile. For example, a stable suspension may be a suspension that has a stable rapid-release fraction and/or a stable in vitro dissolution profile.

A “stable in vitro dissolution profile,” as used herein, refers to an in vitro dissolution profile that is similar considering f₂ factor to in vitro dissolution profile compared to the initial suspension (measured between 1 to 10 days after manufacturing of the suspension, often between 5 to 7 days after manufacturing). For example, a product may be considered stable if it has an f₂ higher than 50 during 6 months at 30° C./65% HR. Other measures of a stable in vitro dissolution profile include having a fraction of the drug released at 30 min that does not differ by more than ±15%, ±10%, or ±5% from the fraction released for the same time in the initial suspension.

### I. Sustained Release Particles

The present disclosure provides a sustained-release (SR) particle that may limit diffusion out of the sustained-release particle when the particle is dispersed in a continuous phase and stored as a suspension for a period of time. For example, the sustained-release particle may include a core, a first coating covering the core, and a second coating covering the first coating. In an embodiment, the core and the first layer may form an intermediate particle. In an embodiment, the sustained-release particles disclosed herein may include a core including guaifenesin, a first layer including one or more hydrophilic polymers, and a second layer including one or more hydrophobic, sustained-release polymers.

The sustained-release particle may have a diameter in the range of about 200 μm to about 500 μm. In some embodiments, the diameter of the core may range from about 200 μm to about 300 μm, about 300 μm to about 400 μm, or about 400 μm to about 500 μm.

The drug-loaded sustained-release particles may be obtained by various techniques known in the art. In some embodiments, the sustained-release particles may be obtained by techniques including, but not limited to, agglomeration in the molten state, such as the Glatt ProCell™ technique, extrusion and spheronization, wet granulation, compacting, granulation and spheronization, where the spheronization is carried out (for example) in a fluidized bed apparatus equipped with a rotor, in particular using the Glatt CPS™ technique, spraying (for example) in a fluidized bed type apparatus equipped with zig-zag filter, in particular using the Glatt MicroPx™ technique, or spraying (for example) in a fluidized bed apparatus optionally equipped with a partition tube or Wurster tube. In an example, drug-loaded sustained-release particles may be obtained by drug layering on neutral cores (such as cellulose spheres) in a fluidized bed. In some examples, the first coating and the second coating may be applied by spraying in a fluidized bed apparatus.

The sustained-release particles may be stable after being stored as a suspension for an extended period of time. The sustained-release particles may be stable after being stored as a suspension for at least 3 months. The sustained-release particles may be stable after being stored as a suspension for at least 6 months. The sustained-release particles may be stable after being stored as a suspension for at least 1 year. The sustained-release particles may be stable after being stored as a suspension for at least 2 years.

For example, the sustained-release particle may have a stable in vitro dissolution profile after storage of the particle as a suspension, for at least three months at about 30° C. and about 65% relative humidity. In other examples, the sustained-release particle may have a stable in vitro dissolution profile after storage of the particle as a suspension, for at least three months at about 25° C. and about 60% relative humidity.

### (a) Core

Each of the sustained-release particles includes a core. The core may be a sphere, particle, pellet, bead, or granule. In some embodiments, the core may include a cellulose sphere, sugar sphere, or a non-pareil seed/bead. Non-limiting examples of the bead or pellet include crystals or spheres of lactose, sucrose (such as Compressuc™ PS from Tereos), microcrystalline cellulose (such as Avicel™ from FMC Biopolymer, Cellet™ from Pharmatrans, Celphere™ from Asahi Kasei, or Vivapur® MCC spheres from JRS Pharma), sodium chloride, calcium carbonate (such as Omyapure™ 35 from Omya), sodium hydrogen carbonate, dicalcium phosphate (such as Dicafos™ AC 92-12 from Budenheim) or tricalcium phosphate (such as Tricafos™ SC93-15 from Budenheim); composite spheres or granules, for example sugar spheres comprising sucrose and starch (such as Suglets™ from NP Pharm), spheres of calcium carbonate and starch (such as Destab™ 90 S Ultra 250 from Particle Dynamics) or spheres of calcium carbonate and maltodextrin (such as Hubercal™ CCG4100 from Huber); or combinations thereof. The core may also comprise other particles of pharmaceutically acceptable excipients such as particles of hydroxypropyl cellulose (such as Klucel™ from Aqualon Hercules), guar gum particles (such as Grinsted™ Guar from Danisco), xanthan particles (such as Xantural™ 180 from CP Kelco). According to specific embodiments, the core is a cellulose microsphere, such as Cellets™ 90, Cellets™ 100, or Cellets™ 127 marketed by Pharmatrans, or also Celphere™ CP 203, Celphere™ CP305, Celphere™ SCP 100.

In various embodiments, the core includes guaifenesin. In an embodiment, the core includes guaifenesin layered onto a bead or a pellet, forming a drug-containing core. In one embodiment, the drug-containing core is a microcrystalline cellulose sphere layered with guaifenesin.

The core may optionally include a binder. In some embodiments, the binder may be in an amount of less than about 5% w/w compared with guaifenesin amount in the core. In an embodiment, the binder may be in an amount of less than about 3% w/w compared with guaifenesin amount in the core. In an embodiment, the binder may be in an amount of less than about 2% w/w compared with guaifenesin amount in the core. In an embodiment, the binder may be in an amount of less than about 1% w/w compared with guaifenesin amount in the core. In an embodiment, the core may not include a binder.

Non-limiting examples of suitable binders include a cellulose derivative, polyvinylpyrrolidone, maltodextrin, sodium alginate, gelatin, starch, a polyacrylamide, polyvinyloxoazolidone, a polyvinylalcohol, a C₁₂-C₁₈ fatty acid alcohol, polyethylene glycol, or a polyol. In an embodiment, the cellulose derivative may be hydroxypropylcellulose or hydroxypropylmethylcellulose.

The core may have a mean diameter in the range of about 50 μm to about 500 μm. In some embodiments, the diameter of the core may range from about 50 μm to about 100 μm, about 100 μm to about 300 μm, about 200 μm to about 400 μm, or about 300 μm to about 500 μm. In one embodiment, the core has a mean diameter of 200 μm to 400 μm.

### (b) First Coating and Intermediate Particle

Each of the sustained-release particles may further include a first coating over the core, forming an intermediate particle. In various embodiments, the first coating includes one or more hydrophilic polymers.

In an embodiment, the first coating may include about 90 wt % or more of one or more hydrophilic polymers. In an embodiment, the first coating may include about 95 wt % or more of the one or more hydrophilic polymers. In another embodiment, the first coating may include about 98 wt % or more of the one or more hydrophilic polymers. In one embodiment, the first coating may consist of one or more hydrophilic polymers.

In various embodiments, one or more of the hydrophilic polymers of the first coating may include polyvinyl alcohol or a cellulose derivative. Examples of cellulose derivatives include hydroxypropyl cellulose, hydroxypropyl methyl cellulose, cellulose acetate phthalate, cellulose acetate succinate, hydroxypropyl methyl cellulose phthalate, carboxymethylethyl cellulose, cellulose acetate trimellitate, and hydroxypropyl methyl cellulose acetate succinate. In one embodiment, the first coating consists of polyvinyl alcohol.

The first coating may cover the core with a coating ratio of about 8 wt % to about 20 wt % of the intermediate particle. In an embodiment, the first coating may cover the core with a coating ratio of at least about 5 wt % of the intermediate particle. In an embodiment, the first coating may cover the core with a coating ratio of at least about 10 wt % of the intermediate particle. In an embodiment, the first coating may cover the core with a coating ratio of at least about 20 wt % of the intermediate particle. In an embodiment, the first coating may cover the core with a coating ratio of up to about 15 wt % of the intermediate particle. In one embodiment, the first coating may cover the core with a coating ratio of about 10 wt % of the intermediate particle.

The intermediate particle may have a mean diameter in the range of about 100 μm to about 500 μm. In some embodiments, the diameter of the core may range from about 100 μm to about 300 μm, about 200 μm to about 400 μm, or about 300 μm to about 500 μm. In one embodiment, the intermediate particle has a mean diameter of 200 μm to 400 μm.

### (c) Second Coating

Each of the sustained-release particles may further include a second coating over the first coating, forming the sustained release particle. In various embodiments, the second coating includes one or more hydrophobic, sustained-release polymers.

In an embodiment, the second coating may include about 80 wt % or more of one or more hydrophobic, sustained-release polymers. In an embodiment, the second coating may include about 85 wt % or more of one or more hydrophobic, sustained-release polymers. In an embodiment, the second coating may include about 90 wt % or more of one or more hydrophobic, sustained-release polymers. In an embodiment, the second coating may include about 95 wt % or more of one or more hydrophobic, sustained-release polymers.

Non-limiting examples of a sustained-release polymer of the second coating include a cellulose ether derivative, a cellulose ester derivative, or a combination thereof. In an embodiment, the cellulose ether derivative may be ethylcellulose. In an example, the ethylcellulose may have a viscosity range of about 90 to about 110 mPa·s when dissolved at 5% in a solvent mixture of 80% toluene and 20% ethanol and measured at 25° C. by an Ubbelohde viscosimeter. In another embodiment, the cellulose ester derivative may be cellulose acetate butyrate.

The second coating may optionally include a plasticizer. In an embodiment, the second coating may include less than 15 wt % of a plasticizer. In an embodiment, the second coating may include less than 10 wt % of a plasticizer. In an embodiment, the second coating may include less than 5 wt % of a plasticizer. In an embodiment, the second coating may include less than 2 wt % of a plasticizer. In an embodiment, the second coating may not include a plasticizer.

Examples of suitable plasticizers include, without limit, castor oil, cutin, glycerol, a glycerol ester, a phthalate, a citrate, a sebacate, a cetyl alcohol ester, a malonate, triacetin, a butyrate, a succinate, a malate, a fumarate, a benzoate, an azelate, or an adipate. In an embodiment, the glycerol ester may be an acetylated glyceride, glycerol monostearate, glyceryl triacetate, or glycerol tributyrate. In an embodiment, the phthalate may be dibutyl phthalate, diethyl phthalate, dimethyl phthalate, or dioctyl phthalate. In an embodiment, the citrate may be acetyltributyl citrate, acetyltriethyl citrate, tributyl citrate, or triethyl citrate. In an embodiment, the sebacate may be diethyl sebacate or dibutyl sebacate. In an embodiment, the malonate may be diethyl malonate. In an embodiment, the succinate may be dibutyl succinate. In an embodiment, the oxalate may be diethyl oxalate. In an embodiment, the fumarate may be diethyl fumarate. In one embodiment, the plasticizer is castor oil.

The second coating may further include a hydrophilic polymer, a surfactant, or any combination thereof. The second coating may optionally include about 0 wt % to about 15 wt % of a hydrophilic polymer. In an embodiment, the second coating may include 15 wt % or less of the hydrophilic polymer. In an embodiment, the second coating may include less than 10 wt % of the hydrophilic polymer. In an embodiment, the second coating may include less than 5 wt % of the hydrophilic polymer. In an embodiment, the second coating may include less than 2 wt % of the hydrophilic polymer. In an embodiment, the second coating may not include a hydrophilic polymer.

Non-limiting examples of the hydrophilic polymer include polyvinylpyrrolidone, a water-soluble cellulose derivative, a polyacrylamide, a poly-N-vinylamide, a poly-N-vinyllactam, or a polyoxyethylene. In one embodiment, the hydrophilic polymer includes polyvinylpyrrolidone.

The second coating may optionally include about 0 wt % to about 5 wt % of a surfactant. In an embodiment, the second coating may include 5 wt % or less of the surfactant. In an embodiment, the second coating may include 2 wt % or less of the surfactant. In an embodiment, the second coating may not include a surfactant.

In an embodiment, the surfactant may be a non-ionic surfactant. Examples of non-ionic surfactants include, but are not limited to, polyoxyl castor oil, and polyoxyl hydrogenated castor oil. In one embodiment, the surfactant includes polyoxyl 40 hydrogenated castor oil.

In one embodiment, the second coating includes about 80 wt % or more of ethyl cellulose, about 0 wt % to about 15 wt % of polyvinylpyrrolidone, about 0 wt % to about 5 wt % of polyoxyl hydrogenated castor oil, and about 0 wt % to about 5 wt % of castor oil.

The second coating covers the first coating to form the sustained-release particle. The coating ratio of the second layer may be about 15 wt % to about 30 wt % of the sustained-release particle. In an embodiment, the coating ratio of the second layer may be at least 15 wt % of the sustained-release particle. In an embodiment, the coating ratio of the second layer may be at least 20 wt % of the sustained-release particle. In an embodiment, the coating ratio of the second layer may be at least 25 wt % of the sustained-release particle. In an embodiment, the coating ratio of the second layer may be at least 30 wt % of the sustained-release particle. In an embodiment, the coating ratio of the second layer may be less than 30 wt % of the sustained-release particle.

II. Pharmaceutical Composition with Sustained Release Particles

Further provided herein is a pharmaceutical composition which includes the sustained release particles in suspension. The sustained-release particles may be formulated and stored as a suspension. For example, the pharmaceutical composition may include a plurality of sustained-release particles dispersed in a liquid phase and guaifenesin in an immediate-release (IR) fraction. In an embodiment, the immediate-release fraction is not contained within a particle. In some embodiments, the immediate-release fraction may include guaifenesin.

In an embodiment, the total amount of guaifenesin in 1 ml of the pharmaceutical composition may be about 60 mg to about 240 mg.

In an embodiment, the amount of guaifenesin may be equally divided between the plurality of sustained-release particles and the immediate-release fraction. In another embodiment, the dose of the guaifenesin may be greater in the plurality of sustained-release particles than in the immediate-release fraction. That is, the plurality of sustained-release particles may include more than about 50% by weight of the guaifenesin. In other embodiments, the guaifenesin in the sustained-release particles may be about 50% to about 90% by weight of the total guaifenesin and the guaifenesin in the immediate release fraction may comprise about 50% to about 10% by weight of the total guaifenesin, respectively. In an embodiment, the guaifenesin in the immediate-release fraction is about 15% to 25% of the total amount of guaifenesin. In one embodiment, the composition includes at least about 10% of the guaifenesin in the form of the immediate-release fraction. In another embodiment, 20 wt % of the total amount of guaifenesin in the composition is in the form of the immediate-release fraction and the remainder is contained in the sustained-release particles.

The total amount of guaifenesin in 1 ml of the composition may be about 60 mg to about 240 mg. In some embodiments, the amount of guaifenesin in 1 ml of the composition is at least about 60 mg. In some embodiments, the amount of guaifenesin in 1 ml of the composition is at least about 120 mg. In some embodiments, the amount of guaifenesin in 1 ml of the composition is at least about 180 mg. In some embodiments, the amount of guaifenesin in 1 ml of the composition is less than about 240 mg. In an embodiment, the amount of guaifenesin in 1 ml of the composition is about 120 mg.

The liquid phase of the composition may further include one or more osmotic agents. Non-limiting examples of an osmotic agent include a salt, a sugar alcohol, citrate, polydextrose, fructose, glucose, maltose, or sucrose. The sugar alcohol may be maltitol, sorbitol, erythritol, xylitol, or combinations thereof.

The amount of the osmotic agent may be about 30 wt % to about 80 wt % of the composition. In an embodiment, the amount of the osmotic agent may be at least about 30 wt % of the composition. In an embodiment, the amount of the osmotic agent may be at least about 40 wt % of the composition. In an embodiment, the amount of the osmotic agent may be at least about 50 wt % of the composition. In an embodiment, the amount of the osmotic agent may be at least about 60 wt % of the composition. In an embodiment, the amount of the osmotic agent may be at least about 70 wt % of the composition. In an embodiment, the amount of the osmotic agent may be less than about 80 wt % of the composition.

The liquid phase of the composition may further include a crystallization inhibitor. Non-limiting examples of a crystallization inhibitor include polyvinylpyrrolidone, polyethylene glycol of low molecular weight (e.g. PEG 400), or poloxamer (e.g. poloxamer 188). In an embodiment, the crystallization inhibitor may be polyvinylpyrrolidone.

The crystallization inhibitor may be present in an amount that is about 0.1 wt % to about 5 wt % of the composition. In an embodiment, the crystallization inhibitor may be present in an amount that is at least about 0.1 wt % of the composition. In an embodiment, the crystallization inhibitor may be present in an amount that is at least about 1 wt % of the composition. In an embodiment, the crystallization inhibitor may be present in an amount that is at least about 2 wt % of the composition. In an embodiment, the crystallization inhibitor may be present in an amount that is at least about 3 wt % of the composition. In an embodiment, the crystallization inhibitor may be present in an amount that is at least about 4 wt % of the composition. In an embodiment, the crystallization inhibitor may be present in an amount that is less than about 5 wt % of the composition.

The liquid phase may further include one or more of a suspending agent, a preservative, a pH modifier, an antimicrobial agent, and a flavor modifying agent.

The suspending agent may be microcrystalline cellulose and carboxymethylcellulose sodium, xanthan gum, sodium alginate, carrageenan, pectin, or a combination thereof. In an embodiment, the suspending agent is a combination of microcrystalline cellulose and carboxymethylcellulose. The amount of the suspending agent may be about 0.1 wt % to about 5 wt % of the composition. In an embodiment, the amount of the suspending agent may be about 0.1 wt % to about 2 wt % of the composition. In an embodiment, the amount of the suspending agent may be about 0.5 wt % to about 2 wt % of the composition.

The preservative may be benzoic acid or a salt thereof, sorbic acid or a salt thereof, benzyl alcohol, methyl paraben, ethyl paraben, propyl paraben, butyl paraben, or a quaternary ammonium salt, alcohol or phenol. In an embodiment, the preservative is sodium benzoate. The amount of the preservative may be about 0.01% to about 1%. In an embodiment, the amount of the preservative may be about 0.1%.

The pH modifier may be citric acid, malic acid, acetic acid, succinic acid, tartaric acid, hydrochloric acid, or sodium hydroxide. In an embodiment, the pH modifier is citric acid. The amount of the pH modifier may be about 0.04% to about 1%. In an embodiment, the amount of the pH modifier may be about 0.085%.

The amount of flavor modifying agent may be in a range of 0% to 1%. Non-limiting examples of a flavor modifying agent include cherry flavor, grape flavor, tutti Frutti flavor, lemon flavor, orange flavor, or bubble gum flavor.

The pharmaceutical composition may be stable after storage for an extended period of time. In various embodiments, the pharmaceutical composition may be stable after storage for at least 1 month, at least 3 months, at least 6 months, at least 1 year, or at least 2 years. The pharmaceutical composition may be stored at a temperature ranging from about 30° C. to about 45° C. and a relative humidity of about 60% to about 65%.

After storage for at least one month at about 30° C. and 65% relative humidity, the composition may have a stable in vitro dissolution profile. After storage for at least three months at about 30° C. and 65% relative humidity, the composition may have a stable in vitro dissolution profile. After storage for at least 6 months at about 30° C. and 65% relative humidity, the composition may have a stable in vitro dissolution profile. After storage for at least 12 months at about 30° C. and 65% relative humidity, the composition may have a stable in vitro dissolution profile. After storage for at least two years at about 30° C. and 65% relative humidity, the composition may have a stable in vitro dissolution profile.

In an embodiment, the dissolution profile of the composition has an F₂ value of greater than 50 after storage for greater than 6 months when compared to the dissolution profile of an initial suspension. In an embodiment, the dissolution profile of the composition has an F₂ value of greater than 60 after storage for greater than 6 months when compared to the dissolution profile of an initial suspension. In an embodiment, the dissolution profile of the composition has an F₂ value of greater than 70 after storage for greater than 6 months when compared to the dissolution profile of an initial suspension. In an embodiment, the dissolution profile of the composition has an F₂ value of greater than 50 after storage for greater than 1 year when compared to the dissolution profile of an initial suspension. In an embodiment, the dissolution profile of the composition has an F₂ value of greater than 60 after storage for greater than 1 year when compared to the dissolution profile of an initial suspension. In an embodiment, the dissolution profile of the composition has an F₂ value of greater than 70 after storage for greater than 1 year when compared to the dissolution profile of an initial suspension. In an embodiment, the dissolution profile of the composition has an F₂ value between about 50 to about 90, about 50 to about 80, about 50 to about 70, about 50 to about 60, about 60 to about 90, about 60 to about 80, about 60 to about 70, about 70 to about 90, about 70 to about 80, or about 80 to about 90 after storage for greater than 6 months when compared to the dissolution profile of an initial suspension. In an embodiment, the dissolution profile of the composition has an F₂ value between about 50 to about 90, about 50 to about 80, about 50 to about 70, about 50 to about 60, about 60 to about 90, about 60 to about 80, about 60 to about 70, about 70 to about 90, about 70 to about 80, or about 80 to about 90 after storage for greater than 1 year when compared to the dissolution profile of an initial suspension.

In various embodiments, a dose of the pharmaceutical composition may be administered to a human subject to treat coughing, symptoms of coughing, nasal discharge, congestion or sneezing associated with a cold, flu or an allergy.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the disclosure. Therefore, all matter set forth or shown in the accompanying drawings is to be interpreted as illustrative and not in a limiting sense.

### Example 1

Sustained-release compositions of guaifenesin were produced that are stable after storage for at least 6 months. The compositions have an immediate-release fraction and a sustained-release fraction and comprise a plurality of particles suspended in a liquid phase. 20% of the total amount of guaifenesin in the composition (by weight) was directly added as crystals in the liquid phase and the remainder was contained in the SR particles. The compositions are therefore referred to as a 20% IR/80% SR composition. The SR particles of the compositions have an inert core coated with a drug layer (referred to as “the drug-containing core” or the “guaifenesin-containing core”), a first layer covering the drug-containing core, and a second layer covering the first layer. The two compositions differ in the coating ratio of the second layer (e.g. 15 wt % or 20 wt %). Further details are provided below and in Tables 1 and 2.

TABLE 1
Guaifenesin SR particles of Example 1: (1) no binder in the drug-
containing core; (2) first layer = PVA, CR 10%; (3) second
layer = EC100/polyvinylpyrrolidone/Kolliphor RH40/CO (89/3/4/4).

| | | | 15 wt % | 20 wt % |
|---|---|---|---|---|
| | | | SR CR | SR CR |
| | | | % wt in particles | |
| Neutral core | Microcrystalline | Cellets ® 127/USP | 11.5 | 10.8 |
| | cellulose | | | |
| Drug layer | guaifenesin | Guaifenesin/USP | 65.0 | 61.2 |
| PVA coating | polyvinyl alcohol | PVA 4-88/ | 8.5 | 8.0 |
| | | Emprove ®/USP | | |
| Sustained- | ethylcellulose | Ethocel ® 100 | 134. | 17.8 |
| release | | Premium/USP | | |
| (SR) coating | polyvinyl- | Plasdone ® | 0.5 | 0.6 |
| | pyrrolidone | K29-32/USP | | |
| | polyoxyl 40 | Kolliphor ® | 0.6 | 0.8 |
| | hydrogenated | RH40/USP | | |
| | castor oil | | | |
| | castor oil | Castor Oil/USP | 0.6 | 0.8 |
| | | | 100 | 100 |

TABLE 2
Composition of Example 1

| | |
|---|---|
| 20 wt % SR CR | 15 wt % SR CR |
| microparticles | microparticles |

% w/w of total composition

| | | |
|---|---|---|
| Water | 14.25 | 14.25 |
| Avicel RC591 | 0.75 | 0.75 |
| Neosorb 70/70 | 69.7 | 70.4 |
| Sodium chloride | 0.45 | 0.45 |
| Sodium benzoate | 0.1 | 0.1 |
| Sucralose powder | 0.1 | 0.1 |
| Citric acid anhydrous | 0.085 | 0.085 |
| Xantural 180 | 0.085 | 0.085 |
| Guaifenesin | 1.92 | 1.92 |
| SR Particles | 12.55 | 11.82 |
| Grape Flavor | 0.05 | 0.05 |
| Total | 100 | 100 |

To manufacture the guaifenesin-containing cores, 1700 g of guaifenesin were dissolved in water heated at 45° C. Drug was layered onto Cellet 127 microcrystalline cellulose cores in a Glatt GPC1.1 fluid bed coater using the Wurster process to produce a drug layer of 85% w/w. The layering conditions were controlled at a product temperature of 31° C., air flow of 50 m3/h, nozzle pressure of 2.7 bars and mean spray rate of 21 g/min.

A first coating consisting of polyvinyl alcohol (PVA) was then applied to the guaifenesin cores to produce an intermediate particle. 100 g of PVA 4-88 was dissolved in water. The film was coated onto 900 g of previously prepared guaifenesin cores in a Glatt GPC1.1 fluid bed coater using the Wurster process to produce a coating ratio of 10 wt %. The coating conditions were controlled at product temperature of 50-51° C., air flow of 80 m3/h, nozzle pressure of 3.7 bars and mean spray rate of 3.7 g/min.

A sustained-release coating containing ethylcellulose/polyvinylpyrrolidone/polyoxyl hydrogenated castor oil/castor oil (89/3/4/4) was then applied to the intermediate particle to produce the final SR particle. The SR coating was applied to 860 g of previously prepared intermediate particles in a Glatt GPCG1.1 fluid bed coater using the Wurster process to produce a coating ratio of 15 wt % or 20 wt %. The coating conditions were controlled at product temperature of 40-41° C., air flow of 50 m3/h, nozzle pressure of 2.5-3.0 bars and mean spray rate of 13.4 g/min.

Prior to preparing suspensions of the final particles, the liquid phase was prepared by mixing water and Avicel RC591 with an appropriate high shear mixing equipment. Neosorb 70/70, sodium chloride, sodium benzoate, sucralose powder, citric acid anhydrous, Xantural 180, Guaifenesin, and grape flavor were then added under stirring in the amounts found in Table 2 until solubilization of the excipients. Final suspensions were obtained by mixing 1312 g of the liquid phase with 188 g of the final SR particles with a coating ratio at 20% and by mixing 1322.8g of liquid phase with 177.2 g of the final SR particles with a coating ratio at 15%. Transfer of the suspension into PET bottles was performed under moderate and continuous stirring. The pH of the suspensions was measured to be about 4.2.

After 5 to 7 days of rest at 25° C. the t=0 analysis of the suspension was performed as described below. The compositions were then stored at 25° C. with 60% relative humidity and at 30° C. with 65% relative humidity (for particles with 15 or 20 wt % SR coating) for evaluation of the stability.

At different times, the stability of the compositions was evaluated by the in vitro dissolution profile of the composition, and comparing the values to the suspension prior to storage. Briefly, a sample volume of the composition was withdrawn from the container being stored at 25° C. or 30° C. after manual shaking of the bottle. Dissolution profiles were obtained by introducing the sample volume into a USP Type II apparatus equipped with 1 L vessels filled with 900 ml of phosphate buffer at pH6.8. The vessels were maintained at 37° C. and the rotational speed of the paddles set at 100 RPM.

To compare the amount of guaifenesin dissolved at 30 min and the kinetics of the curve regardless of sampling variability and selected bottles, a normalization of curves was performed. (T₂₀ₕ=100%). For example, stability of the IR fraction was evaluated by comparing the % drug released at t=30 min of in vitro testing; a change of ±15%, ±10%, or ±5% may be considered stable. Stability was also evaluated by comparing the slope of the in vitro dissolution profile using f₂ calculations. A product may be considered stable if it has an f₂ higher than 50 during 6 months at 30° C./65% HR.

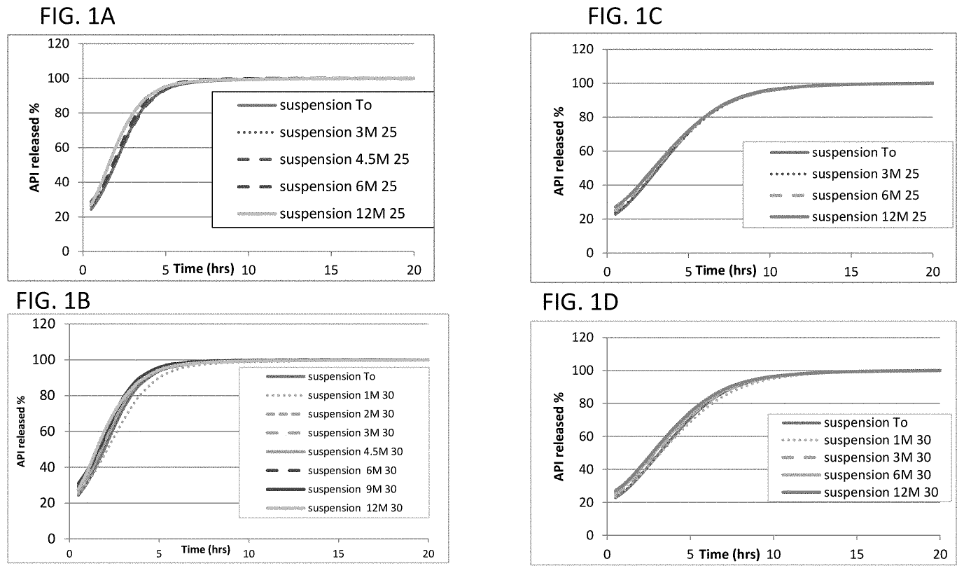

As shown in FIGS. 1A-1D, the compositions of this example have a stable IR fraction and a stable in vitro dissolution profile. Table 3 provides f₂ values corresponding to dissolution profiles of the suspensions stored at 30° C. and 65% relative humidity (FIGS. 1B and 1D).

TABLE 3
F₂ Values

| | 1 month | 2 months | 3 months | 4.5 months | 6 months |
|---|---|---|---|---|---|
| 15 wt % | 67 | 83 | 75 | 72 | 62 |
| SR CR | | | | | |
| 20 wt % | 89 | | 82 | 75 | 72 |
| SR CR | | | | | |

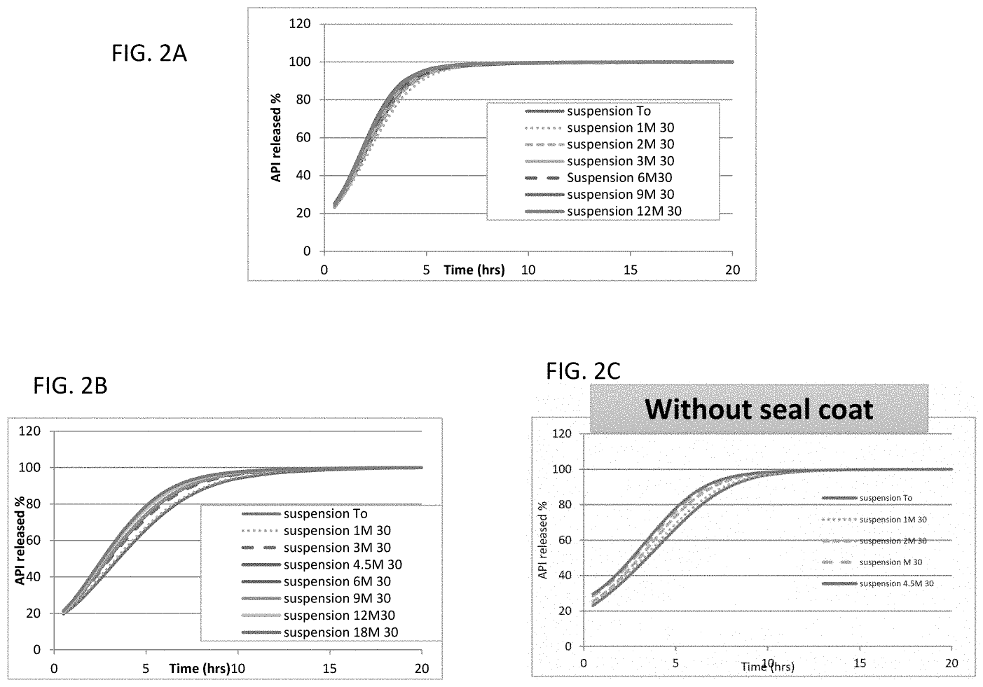

However, compositions comprising particles that lacked a PVA coating, but were otherwise produced as described in this example, are not stable when stored at 30° C. with 65% relative humidity (FIG. 2C).

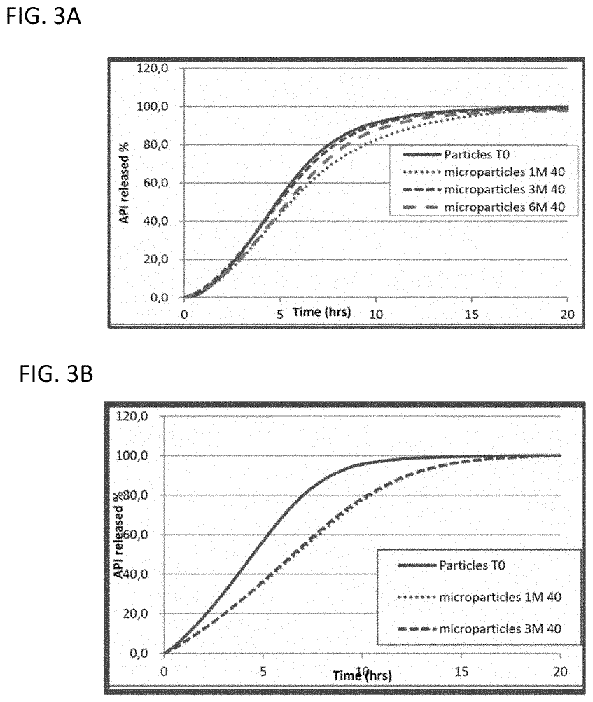

The effect of the PVA coating on the stability of dry particles (20 wt % SR CR) was evaluated. Final particles were prepared as generally described above, with and without a PVA coating, and then stored at 40° C. and 75% relative humidity for various lengths of time. The in vitro dissolution testing conditions were the similar to that described above. Microparticles were lubricated by mixing with 1%(w/w) magnesium stearate. Dry microparticles were weighted to obtain a dose of 300 mg and poured under stirring into the dissolution medium prepared as described above. As shown in FIGS. 3A and 3B, following storage for only one month, particles without a PVA coating were not stable while particles with a PVA coating were stable at least 6 months.

### Example 2

In these experiments, polyvinylpyrrolidone (i.e. povidone) was added to the liquid phase as a crystallization inhibitor (e.g., to prevent the formation/growth of guaifenesin crystals in the liquid phase during storage). Particles and suspension were otherwise prepared as generally described in Example 1. More precisely, PVP was incorporated after dispersion and solubilization of the other excipients in the liquid phase. The mixture was heated at 40° C. before addition of the guaifenesin crystals. After solubilization of the excipients, a cycle of cooling at room temperature was done (below 30° C.), and microparticles were added.

Table 4 provides a detailed description of the amounts of each component in the suspension. Compositions were stored at 30° C. with 65% relative humidity for further evaluation. At different time points, the stability of the compositions was evaluated as described in Example 1.

TABLE 4

| | |
|---|---|
| 20 wt % SR CR | 15 wt % SR CR |
| microparticles | microparticles |

% w/w of total composition

| | | |
|---|---|---|
| Water | 14.25 | 14.25 |
| Avicel RC591 | 0.75 | 0.75 |
| Neosorb 70/70 | 68.7 | 69.4 |
| Sodium chloride | 0.45 | 0.45 |
| Sodium benzoate | 0.1 | 0.1 |
| Sucralose powder | 0.1 | 0.1 |
| Citric acid anhydrous | 0.085 | 0.085 |
| Xantural 180 | 0.085 | 0.085 |
| Plasdone K-12 | 1.00 | 1.0 |
| Guaifenesin | 1.92 | 1.92 |
| Particles | 12.55 | 11.82 |
| Grape Flavor | 0.05 | 0.05 |
| Total | 100 | 100 |

The compositions have a stable IR fraction and a stable in vitro dissolution profile after at least 12 months of storage at 30° C. (FIG. 2A shows 12 months data with particles with a CR of 15 wt %; FIG. 3B shows 12 months data with particles with a CR of 20 wt %).

Table 5 provides f₂ values corresponding to dissolution profiles of the suspensions stored at 30° C. and 65% relative humidity (FIGS. 2A and 2B).

TABLE 5
f₂ Values

| | 1 month | 2 months | 3 months | 4.5 months | 6 months |
|---|---|---|---|---|---|
| 15 wt % | 75 | 93 | 86 | 70 | 72 |
| SR CR | | | | | |
| w/PVA | | | | | |
| 20 wt % | 89 | | 66 | 62 | 60 |
| SR CR | | | | | |
| w/PVA | | | | | |

### Example 3

In these experiments, the guaifenesin-containing core was modified to include a binder. To manufacture the guaifenesin-containing cores, 1615 g of guaifenesin and 85 g of hydroxypropylcellulose (HPC) as a binder were dissolved in water and heated at 45° C. The drug and binder were layered onto Cellet 127 microcrystalline cellulose cores in a Glatt GPC1.1 fluid bed coater using the Wurster process to produce a drug layer of 85% w/w. The layering conditions were controlled at a product temperature of 31° C., air flow of 50 m³/h, nozzle pressure of 3.0 bars and mean spray rate of 23.5 g/min. A PVA coating (CR 10 wt %) was then applied to the guaifenesin cores to produce an intermediate particle and then an ethylcellulose (EC100)/polyvinylpyrrolidone/polyoxyl 40 hydrogenated castor oil (Kolliphor RH40)/castor oil (89/3/4/4; CR 20 wt %) sustained-release coating was applied to the intermediate particle, as generally described in Examples 1 and 2. Particles and suspensions were otherwise prepared as generally described in Example 1. Compositions were stored at 30° C. with 65% relative humidity for further evaluation. At different times, the stability of the compositions was evaluated as described in Example 1.

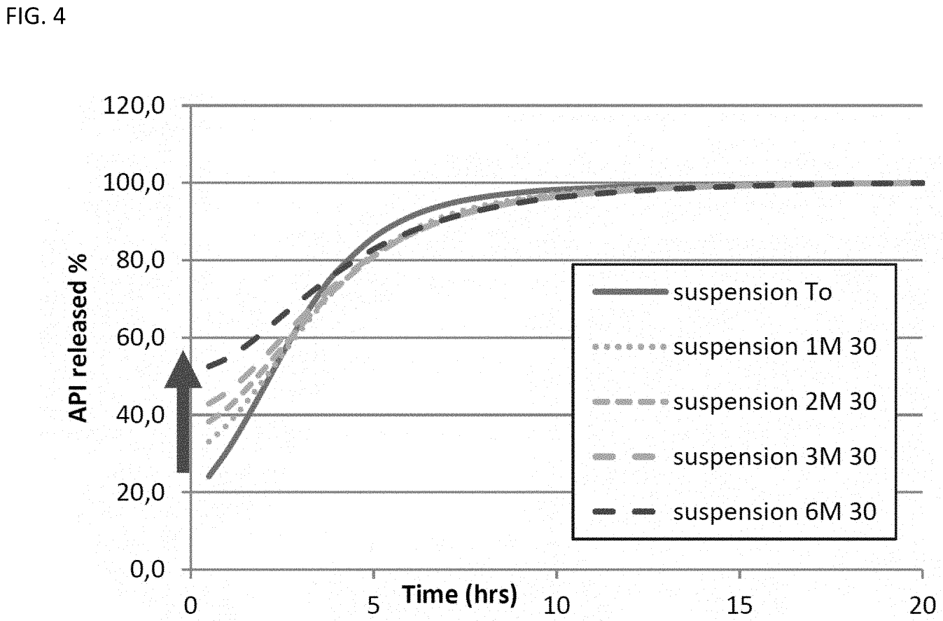

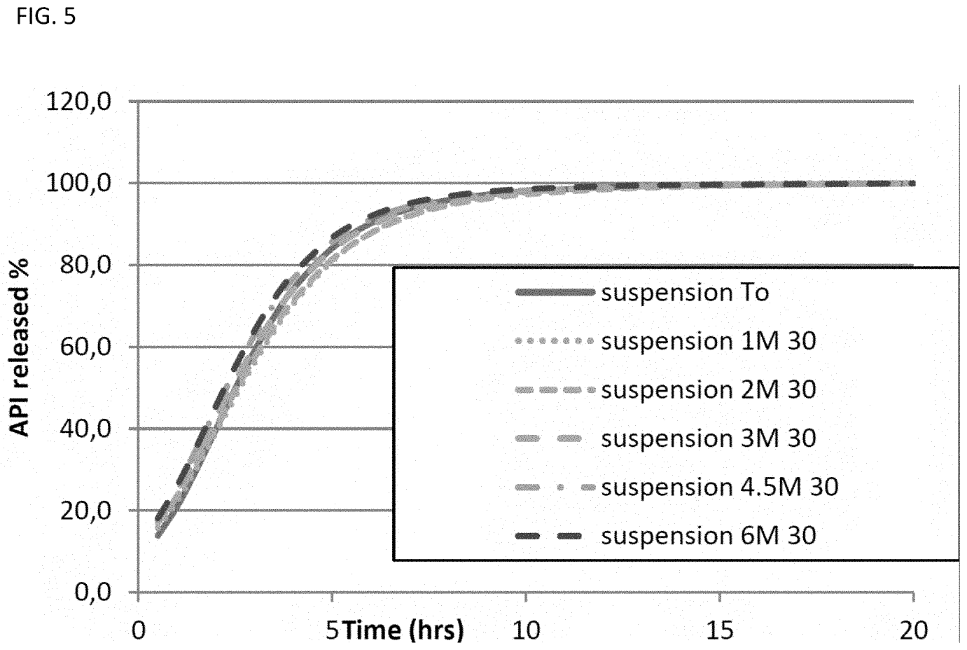

As shown in FIG. 4, the composition was not stable for even one month after storage at 30° C. with 65% relative humidity. The lack of stability was evident, for example, by an increase in the IR fraction after the composition was stored. To evaluate if the concentration of the guaifenesin in the liquid phase affected stability, a suspension of final particles was prepared as generally described above except the immediate release concentration of guaifenesin in the liquid phase was decreased to 10%. As shown in FIG. 5, this composition was stable for at least six months at 30° C. with 65% relative humidity. Overall, it was concluded that final particles containing a binder in the core are stable in suspension when the initial concentration of guaifenesin in the liquid phase is below or near the saturation that is without too many crystals (e.g., about 10% IR).

Table 6 provides f₂ values corresponding to dissolution profiles of the suspensions with particles containing an HPC binder and stored at 30° C. and 65% relative humidity (FIGS. 4 and 5).

TABLE 6
f₂ Values

| | 1 month | 2 months | 3 months | 4.5 months | 6 months |
|---|---|---|---|---|---|
| 20% IR/ | 66 | 58 | 52 | | 42 |
| 80% SR | | | | | |
| w/HPC | | | | | |
| 10% IR/ | 79 | 80 | 80 | 69 | 66 |
| 90% SR | | | | | |
| w/HPC | | | | | |

### Example 4

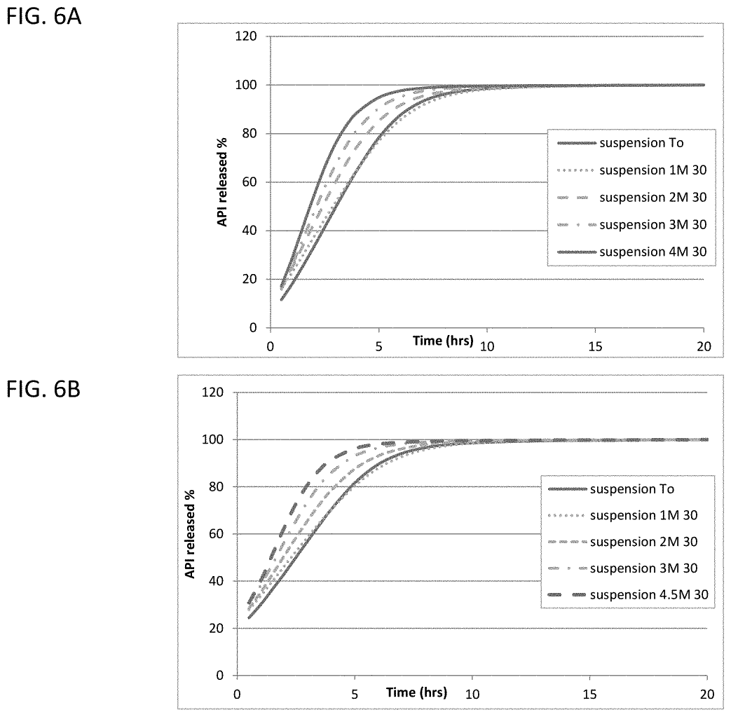

In these experiments, the composition of the SR coating was varied (Ethylcellulose 45P (EC45)/polyvinylpyrrolidone/Kolliphor RH40/CO (80/2/2/16)). The particles also included a PVA seal coat without a binder. Compositions with a 5% IR fraction (FIG. 6A) or a 20% IR fraction (FIG. 6B) were otherwise prepared as generally described in Examples 1 and 2. Compositions were stored at 30° C. with 65% relative humidity for further evaluation. At different time points, the stability of the compositions was evaluated as described in Examples 1 and 2.

As shown in FIGS. 6A and 6B, these particles were found to not be stable for even one month after storage at 30° C. with 65% relative humidity.

Table 7 provides f₂ values corresponding to dissolution profiles of the suspensions with particles with a coating composition of EC45/polyvinylpyrrolidone/Kolliphor RH40/CO (80/2/2/16), a PVA seal coat, and without a binder. The suspensions had a 5% IR fraction (FIG. 6A) or a 20% IR fraction (FIG. 6B) and were stored at 30° C. and 65% relative humidity.

TABLE 6
f₂ Values

| | 1 month | 2 months | 3 months | 4.5 months |
|---|---|---|---|---|
| 5% IR/ | 74 | 53 | 43 | 34 |
| 95% SR | | | | |
| 10% IR/ | 79 | 80 | 80 | 69 |
| 90% SR | | | | |

The lack of stability may be due to high content of the plasticizer castor oil, as compared to the compositions in Examples 1, 2, and 3. Overall, it was concluded that final particles containing a plasticizer in the second coating are stable in suspension when the plasticizer is in an amount of less than about 15 wt % of the second coating.

### Example 5

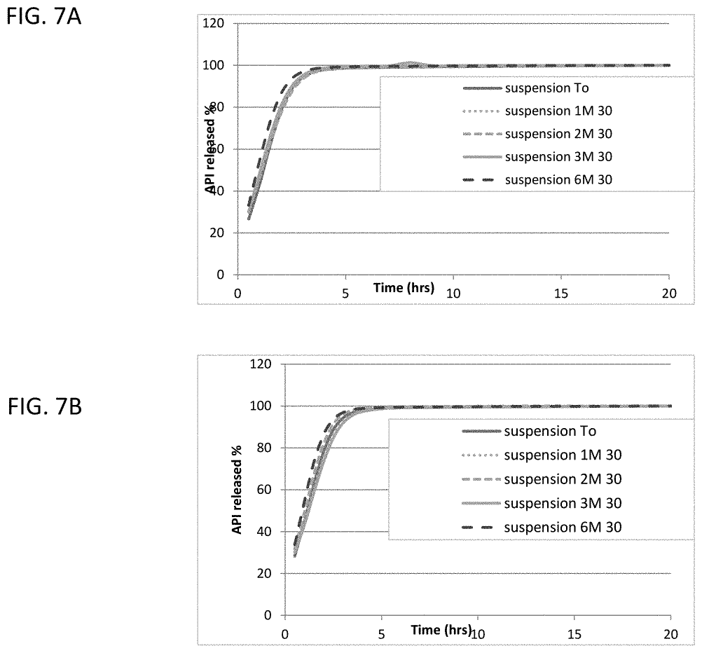

In these experiments, the composition of the SR coating was varied to be EC45/CAB/polyvinylpyrrolidone/Kolliphor RH40/CO (66/19/3/4/8). The particles also included a PVA seal coat without a binder. Compositions with a 20% IR fraction and 1% PVP in the liquid phase (FIG. 7A) or no PVP in the liquid phase (FIG. 7B) were otherwise prepared as generally described in Examples 1 and 2. Compositions were stored at 30° C. with 65% relative humidity for further evaluation. At different time points, the stability of the compositions was evaluated as described in Examples 1 and 2.

As shown in FIGS. 7A and 7B, these particles were found to be stable in suspension for at least 6 months at 30° C. Table 7 provides f₂ values corresponding to dissolution profiles of the suspension with 20% IR fraction and 80% of particles having a coating of composition EC45-CAB/polyvinylpyrrolidone/Kolliphor RH40/CO (66-19/3/4/8) and stored at 30° C. and 65% relative humidity. The suspension either has 1% PVP (FIG. 7A) or no PVP (FIG. 7B).

TABLE 6
f₂ Values

| | 1 month | 2 months | 3 months | 6 months |
|---|---|---|---|---|
| 20% IR/ | 81 | 80 | 71 | 51 |
| 80% SR | | | | |
| w/1% PVP | | | | |
| 20% IR/ | 82 | 70 | 72 | 53 |
| 80% SR | | | | |
| w/HPC | | | | |
| w/o PVP | | | | |

1. A sustained-release particle comprising a core, a first coating, and a second coating, wherein:
the core comprises guaifenesin;
the first coating comprises about 90 wt % or more of one or more hydrophilic polymers and covers the core to form an intermediate particle, wherein the coating ratio of the first coating is about 5 wt % to about 15 wt % of the intermediate particle;
the second coating comprises about 80 wt % or more of one or more hydrophobic, sustained-release polymers, less than about 15 wt % of a plasticizer, and the second coating covers the first coating.

2.-32. (canceled)

33. A pharmaceutical composition comprising a plurality of sustained release particles dispersed in a liquid phase wherein:
the plurality of sustained release particles consists of particles according to claim 1;
the composition comprises at least about 10% of the guaifenesin in the form of an immediate-release (IR) fraction; and
the amount of guaifenesin in 1 ml of the composition is about 60 mg to about 240 mg.

34. The pharmaceutical composition of claim 33, wherein the amount of guaifenesin in 1 ml of the composition is about 120 mg.

35. The pharmaceutical composition of claim 33, wherein the guaifenesin in the IR fraction is about 5% to 50% of the total amount of guaifenesin.

36. The pharmaceutical composition of claim 35, wherein the guaifenesin in the IR fraction is about 15% to 25% of the total amount of guaifenesin.

37. The pharmaceutical composition of claim 33, wherein the liquid phase further comprises one or more osmotic agent and/or crystallization inhibitor.

38. The pharmaceutical composition of claim 37, wherein an osmotic agent is a salt, a sugar alcohol, citrate, polydextrose, fructose, glucose, maltose, or sucrose.

39. The pharmaceutical composition of claim 38, wherein the sugar alcohol is maltitol, sorbitol, erythritol, xylitol, or combinations thereof.

40. The pharmaceutical composition of claim 37, wherein the amount of the osmotic agent is about 30 wt % to about 80 wt % of the composition.

41. The pharmaceutical composition of claim 37, wherein the crystallization inhibitor is polyvinylpyrrolidone, optionally in an amount that is about 0.1 wt % to about 5 wt % of the composition.

42. The pharmaceutical composition of claim 33, wherein the liquid phase further comprises one or more of a suspending agent, a preservative, a pH modifier, an antimicrobial agent, and a flavor modifying agent.

43. The pharmaceutical composition of claim 42, wherein the amount of the suspending agent is about 0.1 wt % to about 5 wt % of the composition, preferably about 0.1 wt % to about 2 wt % of the composition, more preferably about 0.5 wt % to about 2 wt % of the composition.

44. The pharmaceutical composition of claim 42, wherein the suspending agent is microcrystalline cellulose and carboxymethylcellulose sodium, xanthan gum, or a combination thereof.

45. The pharmaceutical composition of claim 42, wherein the preservative is benzoic acid or a salt thereof, sorbic acid or a salt thereof, benzyl alcohol, methyl paraben, ethyl paraben, propyl paraben, butyl paraben, or a quaternary ammonium salt, alcohol or phenol.

46. The pharmaceutical composition of claim 36, wherein after storage for at least three months at about 30° C. and 65% relative humidity, the composition has a stable in vitro dissolution profile.

47. The pharmaceutical composition of claim 46, wherein the composition has a stable in vitro dissolution profile for at least 6 months.

48. The pharmaceutical composition of claim 46, wherein the composition has a stable in vitro dissolution profile for at least 12 months.

49. The pharmaceutical composition of claim 46, wherein the composition has a stable in vitro dissolution profile for at least 2 years.

50. The pharmaceutical composition of claim 46, wherein the stable in vitro dissolution profile is evaluated by comparing a fraction of guaifenesin released at 30 min to an initial suspension.

51. The pharmaceutical composition of claim 50, wherein the fraction of the guaifenesin released at 30 min does not differ by more than ±10% from the fraction released for the same time in the initial suspension.

52. The pharmaceutical composition of claim 46, wherein the stable in vitro dissolution profile is evaluated by calculating an f₂ value.

53. The pharmaceutical composition of claim 52, wherein the f₂ value is greater than 50.

54. A method of treating coughing, symptoms of coughing, nasal discharge, congestion or sneezing associated with a cold, flu or an allergy comprising administering to a human subject in need thereof a single dose of a pharmaceutical composition of claim 33.
